(19) United States
(12) Patent Application Publication
(10) Pub. No.: US 2025/0380959 A1
(43) Pub. Date: Dec. 18, 2025

(54) RETRIEVAL DEVICES AND RELATED SYSTEMS AND METHODS

(71) Applicant: Boston Scientific Scimed, Inc., Maple Grove, MN (US)

(72) Inventors: Laura Emily SABBAN, Norton, MA (US); Camron HAGEMEYER, Bloomington, IN (US); Julian FUENTES CASTRO, La Unión (CR); Cristopher OVIEDO MIRANDA, Alajuela (CR); Gian Franco LOO FUCHS, San Jose (CR); Vivian HERNANDEZ, Heredia (CR)

(73) Assignee: Boston Scientific Scimed, Inc., Maple Grove, MN (US)

(21) Appl. No.: 19/234,500

(22) Filed: Jun. 11, 2025

Related U.S. Application Data
(60) Provisional application No. 63/659,959, filed on Jun. 14, 2024.

(51) Int. Cl. A61B 17/221

(52) U.S. Cl. CPC A61B 17/221; A61B 2017/2215

(57) ABSTRACT

Medical devices, systems, and related methods for capturing objects within a subject (e.g., patient) are described. The medical device includes a sheath, a shaft, and a retrieval device coupled to a distal end of the shaft. At least a portion of the shaft is movably disposed within the sheath. The retrieval device includes a plurality of wires coupled to the distal end of the shaft. The retrieval device is movable between at least a closed state and an open state. Movement of one of the sheath and the retrieval device relative to another one of the sheath or the retrieval device is configured to break at least one wire of the plurality of wires.

### CROSS-REFERENCE TO RELATED APPLICATIONS

This application claims the benefit of priority to U.S. Provisional Application No. 63/659,959, filed on Jun. 14, 2024, which is incorporated by reference herein in its entirety.

### TECHNICAL FIELD

Various aspects of the disclosure relate generally to retrieval devices, systems, and related methods. Examples of the disclosure relate to breakable retrieval devices, systems, and related methods, for example, concerning a medical instrument with an expandable basket or expandable element, with at least a portion of the expandable basket or expandable element being breakable, among other aspects.

### BACKGROUND

Advancements in medical devices, systems, and related methods, have enabled users to perform increasingly complex medical procedures. One challenge in the field of medical procedures is associated with extracting objects from a patient, such as a biliary stone from a bile duct of the patient. These procedures often require multiple instruments to extract the object, which may cause various procedural issues and/or increase the risk of injury to the patient. In some aspects, the object(s) are captured with an expandable retrieval basket delivered through a sheath or tube, but the object(s) can be too big to be removed from the patient through the sheath or tube. In these aspects, it can be difficult to release the object(s) from the retrieval basket, for example, such that the retrieval basket can be removed from the patient. It would be useful to improve medical extraction devices, systems, and related methods to mitigate the risk of procedural issues and/or injury to the patient during medical procedures that involve extracting objects.

### SUMMARY

Examples of this disclosure relate to, among other things, retrieval devices and related systems and methods.

According to one example, a medical device may include a sheath, a shaft, and a retrieval device coupled to a distal end of the shaft. The shaft may include at least a portion movably disposed within the sheath. The retrieval device may include a plurality of wires coupled to the distal end of the shaft. The retrieval device may be movable between at least a closed state and an open state, may be configured to radially compress into the closed state when positioned within the sheath, and may be configured to radially expand into the open state when positioned outside of the sheath. Movement of one of the sheath and the retrieval device relative to another one of the sheath or the retrieval device may be configured to break at least one wire of the plurality of wires of the retrieval device.

Any medical device or medical system described herein may include any of the following features. The sheath may include a distal portion having a sharp edge configured to break at least one of the plurality of wires in response to movement of the sheath relative to the shaft. The distal portion of the sheath may include a tapered outer surface and an inner surface, in which the tapered outer surface is atraumatic and the inner surface includes the sharp edge opposite the tapered outer surface. The sheath may be configured to translate and rotate relative to the shaft. Rotation of the sheath relative to the shaft may cause the sheath to engage at least one of the plurality of wires of the retrieval device, and proximal translation of the sheath relative to the shaft may break at least one of the plurality of wires of the retrieval device. Proximal translation of the sheath relative to the shaft may cause the sheath to engage at least one of the plurality of wires of the retrieval device, and rotation of the sheath relative to the shaft may break at least one of the plurality of wires of the retrieval device. The sheath may be configured to break a proximal portion of at least one of the plurality of wires of the retrieval device in response to at least one of rotation and translation of the sheath relative to the shaft. The plurality of wires may radially expands into the open state to define a cavity configured to capture an object therein.

At least one of the plurality of wires may include at least one defect positioned thereon. The at least one defect may include at least one of a notch, a ground, a heat melt, a laser melt, or a combination thereof. The at least one defect may include a reduced diameter, and at least one of the plurality of wires may include a first portion having a first diameter, a second portion having a second diameter less than the first diameter, and a transition surface extending between the first portion and the second portion. The transition surface may include at least one of a convex surface, a concave surface, a tapered surface, an angled surface, or a combination thereof. The first portion having the first diameter may be positioned proximal of the second portion having the second diameter relative to the distal end of the shaft.

The medical device may include a cutter tool disposed in the sheath. The cutter tool may be movable relative to the sheath and the shaft. The cutter tool may be configured to break at least one of the plurality of the wires of the retrieval device. The cutter tool may include a sharp edge configured to break at least one of the plurality of wires of the retrieval device in response to movement of the cutter tool relative to the retrieval device.

According to another example, a medical device may include a sheath, a shaft disposed in the sheath, a retrieval device disposed on a distal end of the shaft, and a cutter tool disposed in the sheath. The retrieval device may include a plurality of wires coupled to the distal end of the shaft. The retrieval device may be configured to radially compress into a closed state within the sheath, and to radially expand into an open state outside the sheath. The cutter tool may be movable relative to the sheath and the shaft. The cutter tool may be configured to break a proximal portion of at least one of the plurality of the wires of the retrieval device.

Any medical device or medical system described herein may include any of the following features. The cutter tool may include an elongate member configured to be distally advanced through the sheath, and at least one sharp edge disposed on a distal end of the elongate member. The at least one sharp edge of the cutter tool may be configured to break the proximal portion of at least one of the plurality of wires of the retrieval device in response to proximal translation of the elongate member relative to the sheath. The at least one sharp edge may be configured to deflect radially outward relative to the elongate member for engagement with the proximal portion of at least one of the plurality of wires in response to distal translation of the elongate member distally of the distal end of the sheath.

According to another example, a method to perform a medical procedure may include inserting a medical device into a body cavity, advancing a retrieval device distally through a sheath of the medical device, and capturing an object within the retrieval device. The retrieval device may include a plurality of wires coupled to a distal end of a shaft of the medical device. The method may include rotating the sheath relative to the retrieval device to engage at least one of the plurality of wires of the retrieval device, retracting the sheath proximally relative to the retrieval device to break at least one of the plurality of wires of the retrieval device, and releasing the object from the retrieval device. The method may further include, after releasing the object from the retrieval device, retracting the retrieval device and at least one broken wire thereof proximally through the sheath.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 depicts a medical device, according to aspects of the disclosure.

FIG. 2 depicts another medical device, according to aspects of the disclosure.

FIG. 3 depicts another medical device, according to aspects of the disclosure.

FIG. 4 depicts another medical device, according to aspects of the disclosure.

FIG. 5 depicts another medical device, according to aspects of the disclosure.

FIGS. 6A-6D depict various different portions of the medical device of FIG. 5, according to aspects of the disclosure.

FIGS. 7A and 7B depict another medical device, according to aspects of the disclosure.

FIGS. 8A and 8B depict a distal portion of a medical device, according to aspects of the disclosure.

FIGS. 9A and 9B depict another medical device, according to aspects of the disclosure.

FIGS. 10 and 11 depict methods, according to aspects of the disclosure.

### DETAILED DESCRIPTION

Examples of the disclosure include systems, devices, and methods for providing a medical instrument including a retrieval device having expandable members that are selectively expandable for collecting and removing a target object from a target treatment site within a patient.

As used herein, the term “distal” refers to a portion farthest away from a user when introducing a device into a patient and the term “proximal” refers to a portion closest to the user when placing the device into the subject. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to system, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target treatment site”). Various examples described herein include single-use or disposable medical devices, although some embodiments may include one or more reusable components of the device. Reference will now be made in detail to examples of the disclosure described above and illustrated in the accompanying drawings. Wherever possible, the same reference numbers will be used throughout the drawings to refer to the same or like parts.

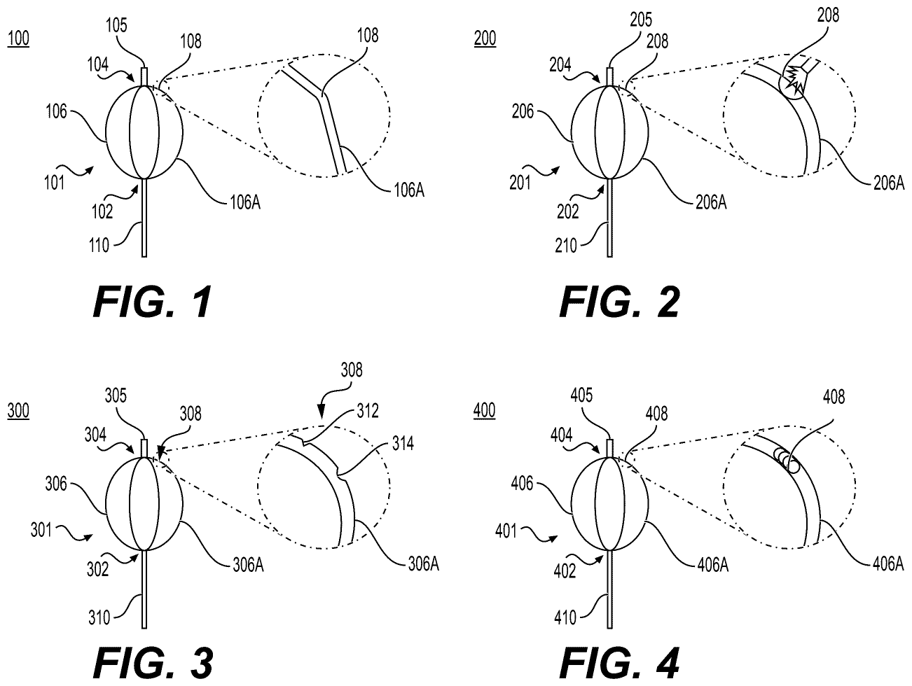

FIG. 1 shows a medical device 100, according to aspects of the disclosure. For example, although not shown, medical device 100 may be delivered through a working channel or other lumen of an endoscope, duodenoscope, gastroscope, colonoscope, ureteroscope, bronchoscope, and/or various other delivery systems to a target treatment site. As shown, the medical device 100 may include a retrieval device 101 coupled to a distal end of a shaft 110. The retrieval device 101 may include a plurality of wires 106 extending between a proximal end 102 and a distal end 104 of the retrieval device 101. As shown, the plurality of wires 106 include proximal ends coupled to the distal end of the shaft 110 at a proximal end 102 of the retrieval device 101, and distal ends coupled to a distal tip 105 at a distal end 104 of the retrieval device 101. In some aspects, one or more of wires 106 may include one or more weak point, defect, or other feature such that the one or more wires 106 may be breakable, for example, to release a captured object.

The medical device 100 may include a handle (not shown) having one or more ports configured to receive and/or control one or more medical instruments therein and/or one or more actuators. The retrieval device 101 may be movable between at least a closed state and an open state. The retrieval device 101 may radially compress into the closed state and radially expand into the open state. For example, the retrieval device 101 may radially compress into the closed state when positioned within a sheath or lumen (not shown) and radially expand when positioned outside of the sheath or lumen. In the open state, the retrieval device 101 may define a cavity for capturing one or more objects therein.

The plurality of wires 106 may include, e.g., one or more wire filaments splitting into multiple wires in a substantially bulbous shape and converging at ends thereof. For example, the plurality of wires 106 may include three, four, five, six, seven, eight or more wires which collectively form the substantially bulbous shape. The plurality of wires 106 may include one or more flexible materials (e.g., Nitinol) that allow the retrieval device 101 to self-expand into the open state (e.g., when positioned outside of or distal to a sheath or lumen). Additionally, or alternatively, one or more portions of the retrieval device 101 may be formed as or otherwise include a net and/or mesh that facilitates retrieval of smaller fragments of material. In other implementations, the retrieval device 101 may be configured to expand manually into the open state.

In some aspects, it may be useful to form at least one defect (e.g., weak point) in one or more portions of the retrieval device 101 of the medical device 100. The defect provides, for example, a reliable break point in the event of object impaction during a medical procedure. For instance, it may be useful to form one or more defects in one or more of the plurality of wires 106 of retrieval device 101. The defect(s) may be disposed on a distal portion of the wire(s), such that the retrieval device reliably breaks at the distal portion. During the medical procedure, in response to object impaction, the wire(s) having the defect(s) may break at the position(s) of defect(s) along the wire(s), and in turn cause the retrieval device 101 to release the object. Forming defects may include, for example, any deformation (e.g., mechanical deformation, chemical deformation, etc.) to structurally weaken at least one portion of the retrieval device (e.g., at least one wire of the plurality of wires). For example, forming the defect may include exposing at least one wire of the retrieval device to at least one of a laser, a heating tool, a rotatable tool, a rigid tool, a kinking tool, or a combination thereof. As such, the defect may include at least one of a notch, a ground, a heat melt, a laser melt, a reduced diameter, or a combination thereof. However, it should be understood that other processes of forming defects may additionally or alternatively be implemented to defect at least one wire of retrieval device. Various methods of forming at least one defect in the retrieval device to provide at least one reliable break point for stone impaction, are discussed herein.

FIGS. 1-4 illustrate different defects in retrieval devices, according to aspects of the disclosure. As shown in FIG. 1, the plurality of wires 106 include at least one wire 106A having a defect 108 positioned thereon. The defect 108 includes a kink point or angular bend, which may be caused by a kinking process yielding plastic deformation and/or micro-cracks in the respective wire of plurality of wires 106. The defect 108 (e.g., kink point) may have lower strength relative to non-kinked points or other portions of the plurality of wires 106, and thus may help to provide a break point to induce failure in the retrieval device 101 (e.g., in the wire 106A) in the event of object impaction during medical procedures.

FIG. 2 shows a medical device 200, according to aspects of the disclosure. The medical device 200 may include a retrieval device 201. Aspects of the retrieval device 201 may be similar to the retrieval device 101 (FIG. 1), the details of which are omitted for brevity. As shown, the retrieval device 201 includes a plurality of wires 206 extending between a proximal end 202 and a distal end 204 of the retrieval device 201. The plurality of wires 206 include proximal ends coupled to a distal end of a shaft 210 at the proximal end 202, and distal ends coupled to a distal tip 205 at the distal end 204. The plurality of wires 206 include at least one wire 206A having a defect 208 formed at a distal portion thereof. As shown, the defect 208 may be a heat induced defect to change structural properties of the retrieval device 201, without substantially modifying the shape and/or dimension of retrieval device 201. Exposing a portion of at least one of the plurality of wires 206 (e.g., wire 206A) to heat over a period of time may yield the defect 208. The exposure temperature, or duration of the exposure period of time, may vary based on the material(s) which form the plurality of wires 206 (e.g., a heat sensitive material). For instance, in some examples, the plurality of wires 206 include Nitinol, and at least one wire 206A is exposed to temperatures upwards of several hundred degrees Celsius for a period of time ranging from several minutes to several hours, which results in precipitation of nickel (Ni) and impacts structural integrity of the exposed portion(s) of the wire 206A. In other examples, one or more of the plurality of wires 206 may include stainless steel or one or more other materials. Additionally, the stainless steel or one or more other materials may be exposed to various temperatures (e.g., for various time durations) to change material properties of the one or more wires 206. For example, the temperature(s) and/or duration(s) of exposure may be configured to reach specific phase transformation temperatures where mechanical properties of the material(s) (e.g., metal(s)) change. The temperature(s) and/or time duration(s) may depend on the material of the respective wires 206.

FIG. 3 shows a medical device 300, according to aspects of the disclosure. The medical device 300 may include a retrieval device 301. Aspects of the retrieval device 201 may be similar to the retrieval device 101 (FIG. 1) and/or the retrieval device 201 (FIG. 2), the details of which are omitted for brevity. As shown, the retrieval device 301 includes a plurality of wires 306 extending between a proximal end 302 and a distal end 304 of the retrieval device 301. The plurality of wires 306 include proximal ends coupled to a distal end of a shaft 310 at the proximal end 302, and distal ends coupled to a distal tip 305 at the distal end 304. The plurality of wires 306 include at least one wire 306A having a defect 308 formed at a distal portion thereof. As shown, the defect 308 may be a mechanically induced deformation, such as a notch 312 or a ground 314. For example, the ground 314 may include a mechanically weakened feature formed by grinding away, or otherwise removing material from, one or more portions of the plurality of wires 306. Forming the defect 308 may include exposing one or more portions of the plurality of wires 306 to at least one mechanical device, such as a rotatable device and/or rigid device. For example, a distal portion of the wire 306A may be exposed to a grinding wheel configured to create the notch 312, and/or exposed to a rigid mechanical fixture configured to create the ground 314. However, other mechanical devices may alternatively or additionally be used to form the defect 308. The portion of the wire 306A that includes the notch 312 or the ground 314 has a reduced thickness relative to unexposed portion(s) (e.g., portions without the defect 308) of the plurality of wires 306. The retrieval device 301 concentrates mechanical stress at the reduced thickness portions (e.g., the defect 308), thus providing one or more reliable break points along the plurality of wires 306 to help induce failure due to object impaction during medical procedures.

FIG. 4 shows a medical device 400, according to aspects of the disclosure. The medical device 400 may include a retrieval device 401. Aspects of retrieval device 401 may be similar to the retrieval device 101 (FIG. 1), the retrieval device 201 (FIG. 2), and/or the retrieval device 301 (FIG. 3), the details of which are omitted for brevity. As shown, the retrieval device 401 includes a plurality of wires 406 extending between a proximal end 402 and a distal end 404 of the retrieval device 401. The plurality of wires 406 include proximal ends coupled to a distal end of a shaft 410 at the proximal end 402, and distal ends coupled to a distal tip 405 at the distal end 404. The plurality of wires 406 include at least one wire 406A having a defect 408 formed at a distal portion thereof. As shown, the defect 408 may be a laser induced defect, for example, resulting from exposure of at least one of the plurality of wires 406 to laser energy. The laser energy may include any laser energy capable of welding material used to form the plurality of wires 406, or otherwise capable of concentrating heat at specific points along a length at least one of the plurality of wires 406A. The portion of the wire 406A exposed to laser energy has a reduced thickness relative to unexposed portions of the wire 406A and/or the other wires 406, and thus may have reduced strength relative to unexposed portions (e.g., portions without the defect 408). The retrieval device 401 concentrates mechanical stress at the reduced thickness portions (e.g., the defect 408), thus providing one or more reliable break points along the plurality of wires 406 to help induce failure due to object impaction during medical procedures.

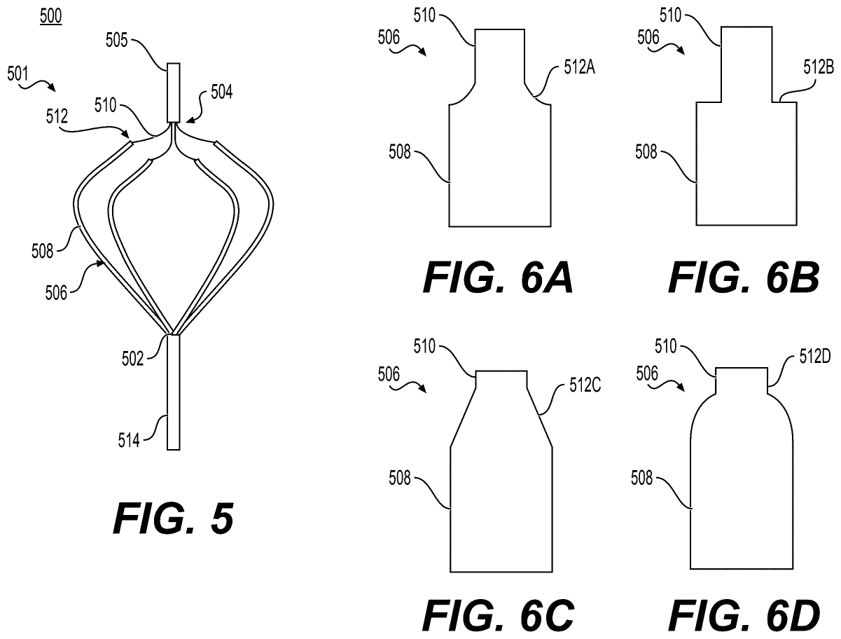

FIG. 5 shows another medical device 500, according to aspects of the disclosure. The medical device 500 may include a retrieval device 501. Aspects of the retrieval device 501 may be substantially similar to aspects of the retrieval device 101 (FIG. 1), retrieval device 201 (FIG. 2), the retrieval device 301 (FIG. 3), and/or the retrieval device 401 (FIG. 4), the details of which are omitted for brevity. As shown, the retrieval device 501 includes a plurality of wires 506 extending between a proximal end 502 and a distal end 504 of the retrieval device 501. The plurality of wires 506 include proximal ends coupled to the distal end of a shaft 514 at the proximal end 502, and distal ends coupled to a distal tip 505 at the distal end 504. Like other retrieval devices discussed herein, the retrieval device 501 may include one or more weak points or sections along a length of at least one of the plurality of wires 506. For example, at least one wire of the plurality of wires 506 may include a variable diameter along a length thereof, for example, between the proximal end 502 and the distal end 504 (e.g., a reduced diameter between two or more portions of wire). In some aspects, as shown in FIG. 6, each wire of the plurality of wires 506 may include the variable diameter.

For example, one or more wires of plurality of wires 506 may include a first portion 508 of the wire 506 having a first diameter, a second portion 510 of the wire 506 having a second diameter different than the first diameter, and a transition portion or surface 512 extending between the first portion 508 and the second portion 510 of the wire 506. As shown, first diameter of the first portion 508 of the wire 506 is greater than the second diameter of the second portion 510 of the wire 506. It should be understood that the plurality of wires 506 may include one or more wires in which the second diameter of the second portion 510 of the wire 506 is greater than or equal to the first diameter of the first portion 508 of the wire 506. Regardless of the specific position(s) of reduced diameter (e.g., at least one defect) along one or more of plurality of wires 506, the retrieval device 501 may help to concentrate stress at the position(s) of reduced diameter, and thereby providing at least one defect to help induce failure of the retrieval device 501 in the event of object impaction during the medical procedure.

It is noted that FIG. 5 illustrates each of wires 506 including the first portion 508 and the second portion 510, with the second portion 510 having a reduced diameter relative to the first portion 508. Nevertheless, this disclosure is not so limited. For example, only one or a subset of the wires 506 may include the reduced diameter between the first portion 508 and the second portion 510.

In some aspects, forming at least one wire having the reduced diameter may include joining (e.g., gluing, soldering, welding, etc.) two or more wires of different diameters together at one or more transition surfaces. For example, welding a distal end of the first portion 508 of the wire 506 to a proximal end of the second portion 510 of the wire 506 at the transition surface 512.

In other aspects, forming at least one wire having the reduced diameter may include grinding or otherwise shaping the second portion 510 of the wire 506 with a mechanical structure (e.g., a grinding wheel, sandpaper, etc.) until a desired diameter is achieved (e.g., second diameter less than the first diameter). The shape and dimension of the transition surface 512 may be controlled during the grinding or shaping process. The transition surface 512 may have any shape and dimension capable of extending between the first portion 508 and the second portion 510 of the wire 506. For example, the transition surface 512 may include at least one of a convex surface, a concave surface, a tapered surface, an angled surface, or a combination thereof. The shape and dimension of transition surface 512 may help to determine, control, or otherwise influence the concentration of stress on the plurality of wires 506 during use, which may be optimized to induce failure at a particular force threshold.

FIGS. 6A-6D illustrate side views of various non-limiting examples of the transition surface 512 of the retrieval device 501, according to aspects of the disclosure. FIG. 6A shows a portion of the wire 506, including a transition surface 512A that includes a concave surface that extends between the first portion 508 and the second portion 510 of the wire 506. The transition surface 512A has a radius of curvature that may be adjusted to modify concentration of stress on the plurality of wires 506. In this aspect, the wire 506 may transition from the diameter of the first portion 508 to the diameter of the second portion 510 in a curved transition.

FIG. 6B shows a transition surface 512B of the retrieval device 501 including a planar surface that extends between the first portion 508 and the second portion 510 of the wire 506. The transition surface 512B may be oriented at an angle relative to the first portion 508 and/or the second portion 510 of the wire 506. For example, the transition surface 512B may be oriented at a ninety degree (90°) angle relative to the first portion 508 and the second portion 510 of the wire 506. In this aspect, the wire 506 may transition from the diameter of the first portion 508 to the diameter of the second portion 510 in a stepwise or distinct transition.

FIG. 6C shows a transition surface 512C of the retrieval device 501 including a tapered surface that extends between the first portion 508 and the second portion 510 of the wire 506. The transition surface 512C has a variable diameter extending from the proximal end of the transition surface 512C to the distal end of the transition surface 512C. For example, as shown, the transition surface 512C may distally extend from the first portion 508 of the wire 506 having the first diameter, and taper radially inward as the transition surface 512C distally extends to the second portion 510 of the wire 506 having the second diameter less than the first diameter. The radially inward taper may be consistent or even throughout a length of the transition surface 512C. In these aspects, the wire 506 may transition from the diameter of the first portion 508 to the diameter of the second portion 510 in a gradual transition.

FIG. 6D shows a transition surface 512D of the retrieval device 501 including a convex surface that extends between the first portion 508 and the second portion 510 of the wire 506. The transition surface 512D has a radius of curvature that may be adjusted to modify concentration of stress on the plurality of wires 506. In this aspect, the wire 506 may transition from the diameter of the first portion 508 to the diameter of the second portion 510 in a curved transition.

Various methods for intentionally breaking one or more basket wires to mitigate basket impaction are discussed herein.

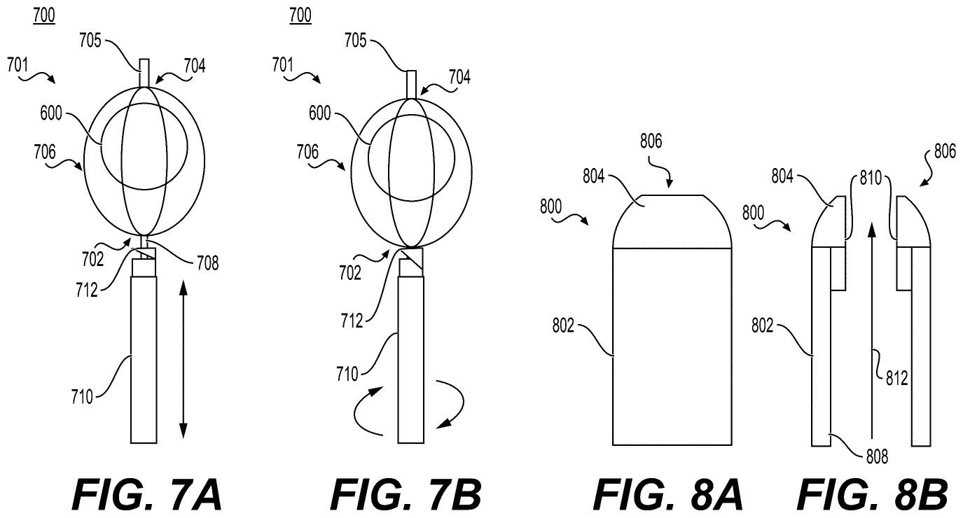

FIGS. 7A and 7B show a medical device 700, according to aspects of the disclosure. The medical device 700 may include a retrieval device 701. Aspects of the retrieval device 701 may be substantially similar to aspects of the retrieval device 101 (FIG. 1), the retrieval device 201 (FIG. 2), the retrieval device 301 (FIG. 3), the retrieval device 401 (FIG. 4), and/or the retrieval device 501 (FIGS. 5-6D), the details of which are omitted for brevity. The retrieval device 701 may include a plurality of wires 706 extending between a proximal end 702 and a distal end 704 of the retrieval device 701. As shown, the plurality of wires 706 include proximal ends coupled to a distal end of a shaft 708 at a proximal end 702 of the retrieval device 701, and distal ends coupled to a distal tip 705 at a distal end 704 of the retrieval device 701. For example, the distal tip 705 may include a hypotube shaped and dimensioned to receive distal ends of the plurality of wires 706 therein, and secured within the distal tip 705 using one or more fixation processes (e.g., crimping, welding, soldering, gluing, etc.).

The medical device 700 may include a handle (not shown) having one or more ports configured to receive and/or control one or more medical instruments therein and/or one or more actuators. The retrieval device 701 may be movable between at least a closed state and an open state. The retrieval device 701 may radially compress into the closed state and radially expand into the open state. For example, the retrieval device 701 may radially compress into the closed state when positioned within a sheath 710 of the medical device 700, and radially expand when positioned outside of the sheath 710. As shown, the shaft 708 may be at least partially disposed in the sheath 710. The shaft 708 and the sheath 710 may move (e.g., translate and/or rotate) relative to each other. For example, the distal end of the shaft 708 may translate distally or proximally relative to the sheath 710, which may cause the retrieval device 701 to translate relative to the sheath 710. In the closed state, the retrieval device 701 may translate distally through the sheath 710 and radially expand (e.g., into the open state) when the distal end of the shaft 708 extends distal of the distal end of the sheath 710. In the open state, the retrieval device 701 may define a cavity for capturing one or more objects therein. For example, the plurality of wires 706 may radially expand to define the cavity in the open state for capturing a stone 600 therein (e.g., a biliary stone in a bile duct).

In some aspects, it may be useful to induce failure in the medical device 700 during a medical procedure such as, for example, to induce failure in at least one of the plurality of wires 706 of the retrieval device 701 and release the stone 600 captured therein. The medical device 700 may induce failure by breaking or cutting at least one of the plurality of wires 706 of the retrieval device 701. The medical device 700 may break at least one of the plurality of wires 706, for example, in response to movement of the shaft 708 relative to the sheath 710 and/or in response to movement of the sheath 710 relative to the shaft 708.

As shown, the sheath 710 may include a sharp edge 712 positioned at the distal end of the sheath 710. The sharp edge 712 may be oriented at an angle relative to a longitudinal axis of the sheath 710. The sharp edge 712 may be shaped and dimensioned to engage and break one or more of the plurality of wires 706 of the retrieval device 701. For example, the sharp edge 712 may engage with and/or help to break one or more of the plurality of wires 706 in response to rotation and/or translation of the sheath 710 relative to the shaft 708, and/or rotation and/or translation of the shaft 708 relative to the sheath 710. In one example, distal translation of the sheath 710 relative to the shaft 708 causes the sharp edge 712 to engage one of the plurality of wires 706, and rotation of the sheath 710 relative to the shaft 708 causes the sharp edge 712 to break the engaged wire(s) of the plurality of wires 706. In another example, rotation of the sheath 710 relative to shaft 708 causes the sharp edge 712 to engage one of the plurality of wires 706, and proximal translation of the sheath 710 relative to the shaft 708 causes the sharp edge 712 to break the engaged wire(s) of the plurality of wires 706. In other examples, the sharp edge 712 may score (e.g., cut or damage partially) one or more of the plurality of wires 706 (e.g., for subsequent retrieval operations to provide a break point to induce failure in retrieval device 701).

FIG. 8A shows a side view of a sheath 800, according to aspects of the disclosure. The sheath 800 may be used with any medical device or medical instrument discussed herein. For instance, the sheath 800 may shaped and dimensioned to receive a retrieval device, such as the retrieval device 701 (FIGS. 7A-7B). As shown, the sheath 800 includes a body 802 extending distally toward a distal tip 804 positioned at a distal end 806 of the sheath 800. The body 802 may extend distally from a handle of a medical device (not shown).

In some aspects, the distal tip 804 of the sheath 800 may be atraumatic or have atraumatic surfaces which, for example, allow atraumatic insertion and/or movement of the sheath 800 into a body lumen of a patient. For instance, in some examples, the distal tip 804 has an outer surface shaped and dimensioned to contact tissue of the patient without causing damage to said tissue, such as a curved or tapered surface. The distal tip 804 may be integrally formed with the body 802 of the sheath 800, or may be attached to the distal portion of the body 802 by, for example, an adhesive, a snap-fit connection, threaded connection, or the like. The distal tip 804 may include an attachment feature (not shown), such as an internal thread or latch, configured for securing the distal tip 804 to and/or radially around the body 802. For instance, the distal tip 804 may include one or more attachment features configured to engage and couple to an external feature of the sheath 800, such as internal thread configured to threadably engage external thread. It should be understood that the attachment feature may additionally or alternatively include other mechanisms to secure the distal tip 804 and the body 802, such as a latch or lock. The distal tip 804 may have variable dimensions at different portions along a length thereof, for example a distally tapering surface shaped and dimensioned for atraumatic contact with tissue of the patient.

FIG. 8B illustrates a longitudinal cross-section view of the sheath 800 of FIG. 8A. As shown, the body 802 of the sheath 800 includes an inner surface 808 defining a lumen 812 extending through the body 802 toward the distal end 806. The lumen 812 of the sheath 800 may include a proximal opening (not shown) shaped and dimensioned to receive one or more medical instruments therein (e.g., shafts, retrieval device, etc.). The medical instrument(s) may be advanced distally through the body 802 toward the distal end 806 of the sheath 800. The lumen 812 of the sheath 800 may include a distal opening formed, for example, in the distal tip 804 at the distal end 806. For example, a retrieval device (e.g., the retrieval device 701 of FIGS. 7A-7B) may move distally through and relative to the sheath 800 (e.g., toward the distal end 806 through the opening formed therein). In some aspects, with relative movement of the sheath 800 and/or the retrieval device, the sheath 800 may engage with and/or break one or more portions of the medical instrument therein (e.g., a proximal portion of the retrieval device 701).

As shown in FIG. 8B, the sheath 800 may include at least one sharp edge 810 disposed in the lumen 812. The sharp edge 810 may be shaped and dimensioned to engage and break one or more portions of the medical instrument disposed within the sheath 800. The sharp edge 810 may be oriented at an angle relative to a longitudinal axis of the sheath 800. In one example, the sharp edge 810 is disposed on the inner surface 808 of the body 802 at the distal end 806 of the sheath 800. In another example, the sharp edge 810 is disposed on an inner surface of the distal tip 804 at the distal end 806 of the sheath 800. For example, the sharp edge 810 may be positioned on an inner surface of the distal tip 804 adjacent the curved or tapered outer surface of the distal tip 804, and oriented toward the lumen 812 of the sheath 800.

In some aspects, the sharp edge 810 may engage a portion of the retrieval device in response to a first movement, and break the retrieval device in response to a second movement different than the first movement. In one example, distal movement of the sheath 800 relative to the retrieval device causes the sharp edge 810 to engage the portion of the retrieval device, and further distal movement of the sheath 800 causes the sharp edge 810 to break the engaged portion of the retrieval device. In another example, proximal movement of the retrieval device relative to the sheath 800 causes the sharp edge 810 to engage and break one or more portions of the retrieval device. For instance, proximal translation of the retrieval device 701 (FIGS. 7A-7B) through the distal end 806 into the sheath 800 causes the sharp edge 810 to engage and break at least one of the plurality of wires 706 of the retrieval device 701. However, it should be understood that other retrieval devices discussed herein may be used in combination with the sheath 800, such as the retrieval device 101 (FIG. 1), the retrieval device 201 (FIG. 2), the retrieval device 301 (FIG. 3), the retrieval device 401 (FIG. 4), and/or the retrieval device 501 (FIGS. 5-6D).

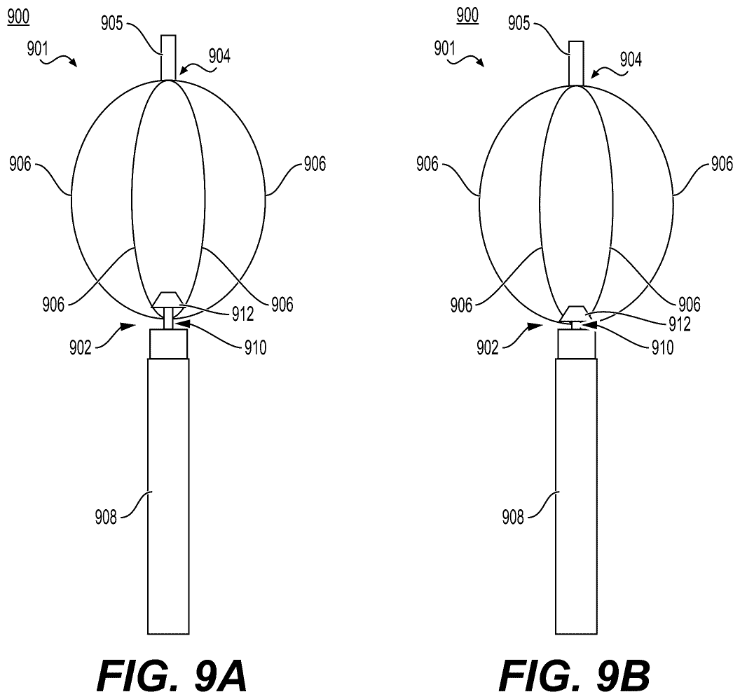

FIGS. 9A and 9B show a medical device 900, according to aspects of the disclosure. The medical device 900 may include a retrieval device 901. Aspects of the retrieval device 901 may be substantially similar to aspects of the retrieval device 101 (FIG. 1), the retrieval device 201 (FIG. 2), the retrieval device 301 (FIG. 3), the retrieval device 401 (FIG. 4), the retrieval device 501 (FIGS. 5-6D), and/or the retrieval device 701 (FIGS. 7A-7B), the details of which are omitted for brevity. The retrieval device 901 may include a plurality of wires 906 extending between a proximal end 902 and a distal end 904 of the retrieval device 901. The plurality of wires 906 include proximal ends coupled to a distal end of a shaft (not shown) at a proximal end 702 of the retrieval device 901, and distal ends coupled to a distal tip 905 at a distal end 904 of the retrieval device 901. The retrieval device 901 may be movable between at least a closed state and an open state. The retrieval device 901 may radially compress into the closed state and radially expand into the open state. For example, the retrieval device 901 may radially compress into the closed state when positioned within a sheath 908 of the medical device 900, and radially expand when positioned outside of the sheath 908. In the open state, the retrieval device 901 may define a cavity for capturing one or more objects therein (e.g., the plurality of wires 906 radially expand into the open state and define the cavity for capturing a biliary stone therein).

In some aspects, it may be useful to induce failure in the medical device 900 during a medical procedure such as, for example, to induce failure in at least one of the plurality of wires 906 of the retrieval device 901 and release one or more objects captured therein. The medical device 900 may include one or more medical instruments to induce failure in one or more portions of the retrieval device 901 such as, for example, to cut a proximal portion of at least one of the plurality of wires 906.

As shown, the medical device 900 may include a cutter tool 910. The cutter tool 910 may include an elongate member (e.g., shaft, tube, or the like) disposed at least partially in the sheath 908. The cutter tool 910 may include a sharp edge 912 disposed on a distal portion of the elongate member. The sharp edge 912 of the cutter tool 910 may be shaped and dimensioned to break one or more portions of the retrieval device 901, such as by cutting the proximal portions of one or more of the plurality of wires 906. The sharp edge 912 may be proximally facing.

The cutter tool 910 may be movable relative to the sheath 908. The cutter tool 910 may be longitudinally movable (e.g., proximally and/or distally) relative to the sheath 908. The cutter tool 910 may also be movable between at least a closed state and an open state. The sharp edge 912 of the cutter tool 910 may deflect or bend radially inward relative to the elongate member into the closed state, and deflect or bend radially outward relative to the elongate member into the open state. For example, the cutter tool 910 may deflect radially inward into the closed state when positioned within the sheath 908, and deflect radially outward when positioned outside of the sheath 908. In the closed state, the cutter tool 910 may move distally through and relative to the sheath 908 (e.g., through a distal opening formed in the distal end of the sheath 908). In the open state, the cutter tool 910 may engage and/or break the retrieval device 901 (e.g., one or more of the plurality of wires 906). In some aspects, the cutter tool 910 may include a compression or biasing member, such as a spring, to bias the sharp edge 912 radially outward relative to the elongate member about a pivot point (e.g., open state of the cutter tool 910).

As shown in FIG. 9A, the cutter tool 910 may extend distally beyond the sheath 908, such that the sharp edge 912 of the cutter tool 910 is positioned distal of the distal end of the sheath 908 within or adjacent to the cavity defined by the retrieval device 901. The sharp edge 912 of the cutter tool 910 may engage a proximal portion of at least one of the plurality of wires 906 of the retrieval device. The sharp edge 912 may cut the proximal portion of the engaged wire(s) in response to movement of the cutter tool 910 relative to the sheath 908. As shown in the FIG. 9B, for example, the cutter tool 910 may translate proximally relative to the sheath 908 (e.g., retract into the sheath 908) and cause the sharp edge 912 to apply force on the engaged wire(s) of the plurality of wires 906, and in turn cut the engaged wire(s) to break the retrieval device 901 (e.g., to release the object captured therein).

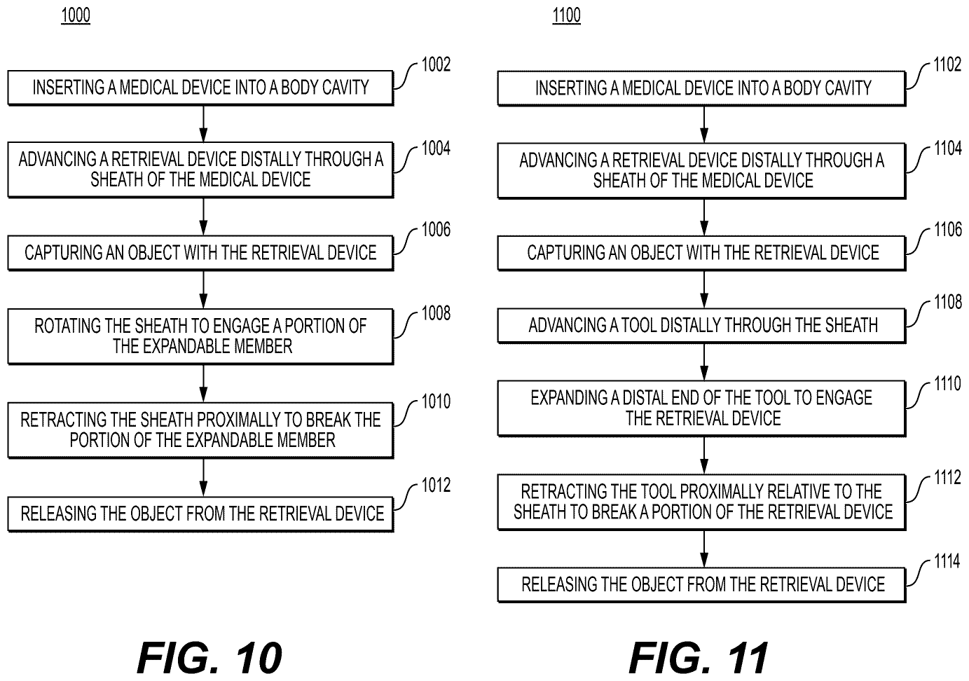

FIG. 10 shows a method 1000 for releasing an object from a retrieval device during a medical procedure, according to aspects of the disclosure. Step 1002 of method 1000 includes inserting a medical device into a body cavity. For example, inserting the sheath 710 of the medical device 700 (FIGS. 7A-7B) into a body lumen of a patient. Step 1004 of method 1000 includes advancing a retrieval device distally through a sheath of the medical device. For example, distally advancing the retrieval device 701 through the sheath 710 (FIGS. 7A-7B).

Step 1006 of method 1000 includes capturing an object with the retrieval device. For example, step 1006 may include capturing the stone 600 with the retrieval device 701 (FIGS. 7A-7B). Step 1008 of method 1000 includes rotating the sheath to engage the retrieval device. For example, rotating the sheath 710 to engage at least one of the plurality of wires 706 of the retrieval device 701 (FIGS. 7A-7B). Step 1010 of method 1000 includes retracting the sheath proximally to break the retrieval device. For example, proximally retracting the sheath 710 relative to the shaft 708 may help to break one or more of the plurality of wires 706 (FIGS. 7A-7B). Step 1012 of method 1000 includes releasing the object from the retrieval device. For example, step 1012 may include releasing the stone 600 from the retrieval device 701 (FIGS. 7A-7B).

FIG. 11 shows a method 1100 for releasing an object from a retrieval device during a medical procedure, according to aspects of the disclosure. Step 1102 of method 1100 includes inserting a medical device into a body cavity. For example, step 1102 may include inserting the sheath 908 of the medical device 900 (FIGS. 9A-9B) into a body lumen of a patient. Step 1104 of method 1100 includes advancing a retrieval device distally through a sheath of the medical device. For example, step 1104 may include distally advancing the retrieval device 901 through the sheath 908 (FIGS. 9A-9B).

Step 1106 of method 1100 includes capturing an object with the retrieval device. For example, step 1106 may include capturing a biliary stone with the retrieval device 901 (FIGS. 9A-9B). Step 1108 of method 1100 includes advancing a cutter distally through the sheath. For example, step 1108 may include distally advancing the cutter tool 910 through and distal of the distal end of sheath 908 (FIGS. 9A-9B). Step 1110 of method 1100 includes engaging a proximal portion of the retrieval device with the cutter. For example, engaging the proximal portion of at least one of the plurality of wires 906 of the retrieval device 901 with the cutter tool 910 (FIGS. 9A-9B). Step 1112 of method 1100 includes retracting the cutter proximally to break the proximal portion of the retrieval device. For example, step 1112 may include retracting the cutter tool 910 proximally relative to the sheath 908 to break the proximal portion of the engaged wire(s) of the retrieval device 901 (FIGS. 9A-9B). Step 1114 of method 1100 includes releasing the object from the retrieval device. For example, step 1114 may include releasing the biliary stone from the retrieval device 901 (FIGS. 9A-9B).

In alternative embodiments, one or more medical devices discussed herein may be electrically active, for example, to transmit current through one or more retrieval devices discussed herein. The electrically active medical device may be monopolar or bipolar. The electrically active medical device may include a retrieval device including one or more non-insulated portions (e.g., not electrically insulated). For example, the retrieval device may have a non-insulated distal tip configured to couple to distal ends of a plurality of wires of the retrieval device. The non-insulated distal tip may degrade when exposed to current, and thereby help to decouple one or more portions of the plurality of wires of the retrieval device. In another example, the retrieval device may have one or more non-insulated wires configured to transmit current to one or more objects captured therein, such as to damage or fragment a captured biliary stone. Further, exposing the retrieval device to current may cause one or more non-insulated wires to break, and in turn release the object therein.

Each of the aforementioned devices, systems, and methods may be used for medical procedures to extract one or more objects from a patient. By providing a medical device capable of able to induce failure in a retrieval device, known problems associated with extraction medical procedures and/or other aspects of invasive surgical procedures may be reduced or avoided. For example, forming a defect in a retrieval device at a known location provides a break or weak point to reliably induce failure in the event of objection impaction on the retrieval device during the extraction procedure. Additionally, providing a medical device capable of breaking the retrieval device during the medical procedure allows for release of large objects that are captured but cannot be safely removed through a body lumen of the patient. By selectively inducing failure at specific locations (e.g., forming defects, cutting, etc.) the retrieval device may break without leaving remnants of the retrieval device inside the body lumen of the patient, which would otherwise damage or harm the patient. In some aspects, inducing failure at a distal portion of the wire may help for the wire to transition to a collapsed configuration when the retrieval device is retracted proximally into a sheath or other insertion device. In other aspects, the wire may be flexible, such that the broken wire may also move within the sheath or other insertion device when the retrieval device is retracted proximally. Based on these aspects, physicians or other users of may reduce the overall procedure time, increase efficiency of procedures, and/or avoid unnecessary harm to a patient's body during medical procedures that involve extracting objects.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a sheath;
a shaft, wherein at least a portion of the shaft is movably disposed within the sheath; and
a retrieval device coupled to a distal end of the shaft, wherein the retrieval device comprises a plurality of wires coupled to the distal end of the shaft,
wherein the retrieval device is movable between at least a closed state and an open state, wherein the retrieval device is configured to radially compress into the closed state when positioned within the sheath, and wherein the retrieval device is configured to radially expand into the open state when positioned outside of the sheath;
wherein movement of one of the sheath and the retrieval device relative to another one of the sheath or the retrieval device is configured to break at least one wire of the plurality of wires of the retrieval device.

2. The medical device of claim 1, wherein the sheath comprises a distal portion having a sharp edge configured to break at least one of the plurality of wires in response to movement of the sheath relative to the shaft.

3. The medical device of claim 2, wherein the distal portion of the sheath comprises a tapered outer surface and an inner surface, wherein the tapered outer surface is atraumatic, and wherein the inner surface includes the sharp edge opposite the tapered outer surface.

4. The medical device of claim 1, wherein the sheath is configured to translate and rotate relative to the shaft.

5. The medical device of claim 4, wherein rotation of the sheath relative to the shaft causes the sheath to engage at least one of the plurality of wires of the retrieval device, and wherein proximal translation of the sheath relative to the shaft breaks at least one of the plurality of wires of the retrieval device.

6. The medical device of claim 4, wherein proximal translation of the sheath relative to the shaft causes the sheath to engage at least one of the plurality of wires of the retrieval device, and wherein rotation of the sheath relative to the shaft breaks at least one of the plurality of wires of the retrieval device.

7. The medical device of claim 4, wherein the sheath is configured to break a proximal portion of at least one of the plurality of wires of the retrieval device in response to at least one of rotation and translation of the sheath relative to the shaft.

8. The medical device of claim 1, wherein the plurality of wires radially expands into the open state to define a cavity configured to capture an object therein.

9. The medical device of claim 1, wherein at least one of the plurality of wires comprises at least one defect positioned thereon.

10. The medical device of claim 9, wherein the at least one defect includes at least one of a notch, a ground, a heat melt, a laser melt, or a combination thereof.

11. The medical device of claim 9, wherein the at least one defect includes a reduced diameter, and wherein at least one of the plurality of wires includes a first portion having a first diameter, a second portion having a second diameter less than the first diameter, and a transition surface extending between the first portion and the second portion.

12. The medical device of claim 11, wherein the transition surface includes at least one of a convex surface, a concave surface, a tapered surface, an angled surface, or a combination thereof.

13. The medical device of claim 11, wherein the first portion having the first diameter is positioned proximal of the second portion having the second diameter relative to the distal end of the shaft.

14. The medical device of claim 1, further comprising a cutter tool disposed in the sheath, wherein the cutter tool is movable relative to the sheath and the shaft, and wherein the cutter tool is configured to break at least one of the plurality of the wires of the retrieval device.

15. The medical device of claim 14, wherein the cutter tool comprises a sharp edge configured to break at least one of the plurality of wires of the retrieval device in response to movement of the cutter tool relative to the retrieval device.

16. A medical device, comprising:
a sheath;
a shaft disposed in the sheath;
a retrieval device disposed on a distal end of the shaft, wherein the retrieval device comprises a plurality of wires coupled to the distal end of the shaft,
wherein the retrieval device is configured to radially compress into a closed state within the sheath, and to radially expand into an open state outside the sheath; and
a cutter tool disposed in the sheath, wherein the cutter tool is movable relative to the sheath and the shaft, and wherein the cutter tool is configured to break a proximal portion of at least one of the plurality of the wires of the retrieval device.

17. The medical device of claim 16, wherein the cutter tool comprises:
an elongate member configured to be distally advanced through the sheath, and
at least one sharp edge disposed on a distal end of the elongate member,
wherein the at least one sharp edge of the cutter tool is configured to break the proximal portion of at least one of the plurality of wires of the retrieval device in response to proximal translation of the elongate member relative to the sheath.

18. The medical device of claim 17, wherein the at least one sharp edge is configured to deflect radially outward relative to the elongate member for engagement with the proximal portion of at least one of the plurality of wires in response to distal translation of the elongate member distally of the distal end of the sheath.

19. A method comprising:
inserting a medical device into a body cavity;
advancing a retrieval device distally through a sheath of the medical device, wherein the retrieval device includes a plurality of wires coupled to a distal end of a shaft of the medical device;
capturing an object within the retrieval device;
rotating the sheath relative to the retrieval device to engage at least one of the plurality of wires of the retrieval device;
retracting the sheath proximally relative to the retrieval device to break at least one of the plurality of wires of the retrieval device; and
releasing the object from the retrieval device.

20. The method of claim 19, further comprising, after releasing the object from the retrieval device, retracting the retrieval device and at least one broken wire thereof proximally through the sheath.
